(19) United States
(12) Patent Application Publication
(10) Pub. No.: US 2020/0155650 A1
(43) Pub. Date: May 21, 2020

(54) Combination treatment of NAFLD and NASH

(71) Applicant: CymaBay Therapeutics, Inc., Newark, CA (US)

(72) Inventors: Yun-Jung Choi, Fremont, CA (US); Charles A. McWherter, Oakland, CA (US)

(73) Assignee: CymaBay Therapeutics, Inc., Newark, CA (US)

(21) Appl. No.: 16/682,408

(22) Filed: Nov. 13, 2019

Related U.S. Application Data
(60) Provisional application No. 62/768,226, filed on Nov. 16, 2018.

(51) Int. Cl. A61K 38/26; A61K 31/192; A61K 9/00; A61P 1/16

(52) U.S. Cl. CPC A61K 38/26; A61P 1/16; A61K 9/0053; A61K 31/192

(57) ABSTRACT

Combination treatment of NAFLD, including NASH, with seladelpar or a salt thereof and a glucagon-like peptide-1 (GLP-1) receptor agonist.

### CROSS-REFERENCE TO RELATED APPLICATIONS

This application claims the benefit under 35 USC 119(e) of Application No. 62/768,226, “Combination treatment of NAFLD and NASH”, filed 16 Nov. 2018, the entire content of which is incorporated into this application by reference.

### FIELD OF THE INVENTION

This invention relates to the combination treatment of non-alcoholic fatty liver disease (NAFLD), including non-alcoholic steatohepatitis (NASH).

### DESCRIPTION OF THE RELATED ART

NAFLD and NASH

Non-alcoholic fatty liver disease (NAFLD) is a disorder affecting as many as 1 in 3-5 adults and 1 in 10 children in the United States, and refers to conditions where there is an accumulation of excess fat in the liver of people who drink little or no alcohol. The most common form of NAFLD is a non-serious condition called hepatic steatosis (fatty liver), sometimes referred to as NAFL, in which fat accumulates in the liver cells: although this is not normal, by itself it is not as concerning as more advanced forms of the disease. NAFL most often presents itself in individuals with a constellation of risk factors referred to as the metabolic syndrome, which includes elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure; but not all patients have all the manifestations of the metabolic syndrome. Obesity is thought to be the most common cause of NAFL, and is also linked to the other risk factors of the metabolic syndrome; and some experts estimate that about two-thirds of obese adults and one-half of obese children may have NAFL. The majority of individuals with NAFL have no symptoms and a normal physical examination, although the liver may be slightly enlarged; children may exhibit symptoms such as abdominal pain and fatigue, and may show patchy dark skin discoloration (acanthosis nigricans). The diagnosis of NAFL is usually first suspected in an overweight or obese person who is found to have mild elevations in their liver blood tests during routine testing, though NAFL can be present with normal liver blood tests, or incidentally detected on imaging investigations such as abdominal ultrasound or CT scan. It is confirmed by imaging studies, most commonly a liver ultrasound or magnetic resonance imaging (MRI), and exclusion of other causes. Unequivocal diagnosis is established by liver biopsy; but biopsy is usually considered warranted only if a number of clinically concerning findings are present.

Some people with NAFL may develop a more serious form of NAFLD called non-alcoholic steatohepatitis (NASH): about 2-5% of adult Americans and up to 20% of those who are obese may suffer from NASH. In NASH, fat accumulation in the liver is associated with inflammation and different degrees of scarring. NASH is a potentially serious condition that carries a substantial risk of progression to cirrhosis, end-stage liver disease, and hepatocellular carcinoma. Some patients who develop cirrhosis are at risk of liver failure and may eventually require a liver transplant. NASH is also associated with cardiovascular events. The most common adverse events in people diagnosed with NASH are cardiovascular: myocardial infarction, angina, stroke, etc., seen in up to 40% of NASH patients; whereas liver-related events occur in approximately 8% of NASH patients.

NASH may be defined within the spectrum of NAFLD by the NAFLD Activity Score (NAS), the sum of the histopathology scores of a liver biopsy for steatosis (0 to 3), lobular inflammation (0 to 2), and hepatocellular ballooning (0 to 2). A NAS of <3 corresponds to non-NASH NAFLD, 3-4 corresponds to borderline NASH, and ≥5 corresponds to NASH. The biopsy is also scored for fibrosis (0 to 4).

NASH, as the extreme form of NAFLD, is a leading cause of end-stage liver disease; while NAFL, and to a greater degree NASH, and the cardiovascular complications associated with them, are intimately related to states of the metabolic syndrome, including insulin resistance (pre-diabetes) and type 2 diabetes mellitus (T2DM), and abdominal obesity. Interventions resulting in weight loss in obese patients, such as lifestyle modification (Vilar-Gomez et al., “Weight Loss Through Lifestyle Modification Significantly Reduces Features of Nonalcoholic Steatohepatitis”, Gastroenterology, 149, 367-378 (2015)) and bariatric surgery (McCarty et al., “Impact of bariatric surgery on outcomes of patients with nonalcoholic fatty liver disease: a nationwide inpatient sample analysis, 2004-2012, Surg. Obes. Relat. Dis., 14, 74-80 (2018), and Tan et al., “Long-term effect of bariatric surgery on resolution of nonalcoholic steatohepatitis (NASH): An external validation and application of a clinical NASH score”, Surg. Obes. Relat. Dis., (2018): https://doi.org/10.1016/j.soard.2018.05.024) have been reported to reduce risk factors in NAFLD and NASH. T2DM has been the most prominent predictor for a poor prognosis in NAFLD. NASH develops much more frequently in the presence of longstanding T2DM, and the majority of patients with cryptogenic cirrhosis are obese and/or diabetic. Studies have demonstrated that 60% of patients with T2DM and NAFLD had biopsy-proven NASH, and that advanced hepatic fibrosis was present in 75% of those with diabetes and hypertension compared to only 7% without either condition. Haukeland, “Abnormal glucose tolerance is a predictor of nonalcoholic steatohepatitis and fibrosis in patients with non-alcoholic fatty liver disease”, Scand. J. Gastroenterol., 40, 1469-1477 (2005), reported that impaired glucose tolerance (IGT) and T2DM were key independent risk factors for severe NAFLD and NASH, increasing the odds ratio almost 4-fold. Mofrad, “Clinical and histological spectrum of nonalcoholic fatty liver disease associated with normal ALT levels”, Hepatology, 37, 1286-1292 (2003), found T2DM to be the only factor independently associated with an increased risk of advanced fibrosis. NASH is understood to be a common complication of T2DM that is frequently associated with fibrosis and which results in cirrhosis in approximately 10% of these patients; and the risk of hepatocellular carcinoma is also increased in patients with T2DM and NASH. Patients with NAFLD (including NASH) usually demonstrate mixed dyslipidemia and the other metabolic derangements described above, including an atherogenic low-density lipoprotein (LDL) phenotype consisting of predominantly of small dense particles. Both metabolic syndrome and NAFLD/NASH are characterized by increased cardiovascular inflammation as measured by elevations in high sensitivity C-reactive protein (hsCRP) and other inflammatory cytokines.

There is significant worldwide incidence of obesity, metabolic syndrome, pre-diabetes and diabetes, with the prevalence of diabetes worldwide predicted to double to 366 million by 2030. The US population with diabetes has been estimated at 25.4 million (11.5% prevalence) in 2011 and 37.7 million (14.5%) by 2031, with 20.2% of Hispanic adults having diabetes. Because approximately 70% of persons with T2DM have a fatty liver, and the disease follows a more aggressive course with necroinflammation and fibrosis (i.e., NASH) in diabetes, the epidemiology of diabetes suggests significant increases in NASH and chronic liver disease. Using MRI for the noninvasive assessment of hepatic steatosis, the prevalence of NAFLD, when defined as liver fat >5%, has been estimated to be 34% in the USA or approximately 80 million people, and as many as two out of three obese subjects. However, this prevalence is believed to be much higher in T2DM. In a series of 107 unselected patients with T2DM, the prevalence of NAFLD by MRI was 76%, which is similar to recent studies from Italy and Brazil. Recent studies have indicated that the prevalence of NAFLD is rapidly rising in obese children and adolescents, especially those of Hispanic ancestry.

Treatments for NAFLD and NASH

There are no drugs currently approved to prevent or treat NAFLD or NASH. A number of pharmacological interventions have been tried in NAFLD/NASH but with overall limited benefit. Antioxidant agents may arrest lipid peroxidation and cytoprotective agents stabilize phospholipid membranes, but agents tried unsuccessfully or with only modest benefit so far include ursodeoxycholic acid, vitamins E (α-tocopherol) and C, and pentoxifylline, among others. Weight-loss agents such as orlistat have had no significant benefit compared to just the use of diet and exercise to achieve weight loss (“weight loss alone”). Most weight-loss studies in NAFLD/NASH have been pilot studies of short duration and limited success, reporting only a modest improvement in necroinflammation or fibrosis. A randomized, double-blind, placebo-controlled 6-month trial (Belfort, “A placebo-controlled trial of pioglitazone in subjects with nonalcoholic steatohepatitis”, N. Engl. J. Med., 355, 2297-2307 (2006)) of weight loss alone against pioglitazone, a thiazolidinedione peroxisome proliferator-activated receptor-γ (PPARγ) agonist and insulin sensitizer, failed to demonstrate any improvement for weight loss alone, but treatment with pioglitazone improved glycemic control, insulin sensitivity, indicators of systemic inflammation (including hsCRP, tumor necrosis factor-α, and transforming growth factor-β), and liver histology in patients with NASH and IGT or T2DM. Treatment with pioglitazone also ameliorated adipose, hepatic, and muscle IR, and was associated with an approximately 50% decrease in necroinflammation (p<0.002) and a 37% reduction in fibrosis (p=0.08). Improvement in hepatocellular injury and fibrosis has been recently reported in another controlled trial with pioglitazone of 12 months duration. In contrast, while the first randomized clinical study with rosiglitazone, the other thiazolidinedione approved for diabetes treatment, in NASH demonstrated a reduction in IR, plasma alanine aminotransferase (ALT) levels and steatosis, rosiglitazone treatment had no significant effect on necrosis, inflammation, or fibrosis. A preliminary report of the 2-year, open-label follow-up of this trial was also disappointing, with no significant benefit from rosiglitazone treatment. Thus, the pharmacological agent with the most robust efficacy in NASH is pioglitazone. Unfortunately, pioglitazone is also associated with a significantly increased risk of weight gain, edema, congestive heart failure, and osteoporotic fractures in both women and men.

According to a report in Markets Insider from 16 Jan. 2018 (http://markets.businessinsider.com/news/stocks/the-race-to find-a-treatment-for-nash-1013102677), 27 companies had compounds in Phase 2 or Phase 3 studies for NASH.

There are three companies with compounds in Phase 3 studies for NASH. Allergan plc (Tobira Therapeutics, Inc.) is evaluating cenicriviroc, a C-C chemokine receptor types 2 and 5 (CCR2/CCR5) antagonist with once-daily oral dosing at 150 mg/day, in the AURORA study (NCT03028740) in patients with NASH and stage 2-3 liver fibrosis according to the NASH Clinical Research Network (CRN) classification. The study has an estimated primary completion date of mid-2019 and study completion date of mid-2024. Genfit SA is evaluating elafibranor, a dual peroxisome proliferator-activated receptor-α/peroxisome proliferator-activated receptor-6 (PPARα/δ) agonist [(EC₅₀ (PPARα)=6 nM; EC₅₀ (PPARδ)=47 nM) agonist with once-daily oral dosing at 120 mg/day (it has also been tested at 80 mg/day), in the RESOLVE-IT study (NCT02704403) in patients with NASH and stages 1-3 liver fibrosis. The study has an estimated primary completion date of mid-2021. Intercept Pharmaceuticals Inc. is evaluating obeticholic acid (OCALIVA, OCA, 6α-ethylchenodeoxycholic acid), a semi-synthetic bile acid analog that is a highly potent farnesoid X receptor (FXR) agonist with once-daily oral dosing at 10 mg/day (or 10 mg/day with titration to 25 mg/day at 3 months), in the REGENERATE study (NCT02548351) in patients with NASH and stages 2-3 (and stage 1 with additional risk factors) fibrosis. The estimated primary and study completion date is late 2022. Obeticholic acid was approved in the US in May 2016 for the treatment of primary biliary cholangitis, in combination with ursodeoxycholic acid.

Twenty-three other companies have compounds in Phase 2, including Inventiva Pharma, with lanifibranor, a pan-PPAR agonist with once-daily oral dosing at 800 and 1200 mg/day; and Novo Nordisk, with semaglutide, a glucagon-like peptide-1 (GLP-1) receptor agonist (incretin mimic), under testing at 0.1, 0.2 and 0.4 mg/day subcutaneous injectable dosing. Liraglutide, another GLP-1 receptor agonist, has also demonstrated activity in NASH at 1.8 mg/day subcutaneous injectable dosing.

GLP-1 Receptor Agonists

GLP-1 receptor agonists are used for the treatment of T2DM. GLP-1 receptor agonists approved in the US include: exenatide (BYETTTA/BYDUREON), approved in 2005/2012 and marketed at 10 μg twice daily (BYETTA) and 2 mg/week (BYDUREON); liraglutide (VICTOZA), approved in 2010 and marketed at 1.2 and 1.8 mg/day, also approved in 2014 for weight loss as SAXENDA and marketed at 3 mg/week; lixisenatide (LYXUMIA), approved in 2016 and marketed at 20 μg/day; dulaglutide (TRULICITY), approved in 2014 and marketed at 0.75 and 1.5 mg/week; and semaglutide (OZEMPIC), approved in 2017 and marketed at 0.5 and 1.0 mg/week. All have subcutaneous injectable dosing. Davies et al., “Effect of Oral Semaglutide Compared With Placebo and Subcutaneous Semaglutide on Glycemic Control in Patients With Type 2 Diabetes: A Randomized Clinical Trial”, JAMA, 318(15), 1460-1470 (2017)), report that semaglutide in a sodium N-[8-(2-hydroxybenzoyl)amino]caprylate (salcaprozate sodium, SNAC) carrier is efficacious in once/day oral dosing at 20 and 40 mg/day; and semaglutide (RYBELSUS) has been approved in the United States and marketed for once/day oral dosing at 7 mg/day, with a run-in at 3 mg/day for 30 days and the option to increase to 14 mg/day for additional glycemic control. From a comparison of the doses marketed for T2DM and tested in NASH, dosing for NASH appears likely to be similar to or somewhat higher than that used for T2DM. “GLP-1 receptor agonists” also include compounds that are dual agonists of glucose-dependent insulinotropic polypeptide (GIP) receptors and GLP-1 receptors, GIP/GLP-1 receptor agonists. An example of this class of compounds, which first emerged in 2013, is tirzepatide (LY3298176): see Coskum et al., “LY3298176, a novel dual GIP and GLP-1 receptor agonist for the treatment of type 2 diabetes mellitus: From discovery to clinical proof of concept”, Mol. Met., (2018), https://doi.org/10.1016/j.molmet.2018.09.009, and Frias et al., “Efficacy and safety of LY3298176, a novel dual GIP and GLP-1 receptor agonist, in patients with type 2 diabetes: a randomised, placebo-controlled and active comparator-controlled phase 2 trial”, Lancet, (2018), http://dx.doi.org/10.1016/S0140-6736(18)32260-8.

Seladelpar

Seladelpar (MBX-8025, (R)-2-(4-((2-ethoxy-3-(4-(trifluoromethyl)phenoxy)propyl)-sulfanyl)-2-methylphenoxy)acetic acid) is an orally active, potent (2 nM) agonist of PPARδ; and is specific, being >600-fold and >2500-fold more potent at the PPARδ receptor than at the PPARα and PPARγ receptors. Seladelpar and its synthesis, formulation, and use is disclosed in, for example, U.S. Pat. No. 7,301,050 (compound 15 in Table 1, Example M, claim 49), U.S. Pat. No. 7,635,718 (compound 15 in Table 1, Example M), and U.S. Pat. No. 8,106,095 (compound 15 in Table 1, Example M, claim 14). Lysine (L-lysine) salts of seladelpar and related compounds are disclosed in U.S. Pat. No. 7,709,682 (seladelpar L-lysine salt throughout the Examples, crystalline forms such as seladelpar L-lysine dihydrate salt claimed).

A Phase 2 study of seladelpar, as the L-lysine dihydrate salt, in mixed dyslipidemia (6 groups, approximately 30 subjects/group: once daily placebo, atorvastatin 20 mg, or seladelpar L-lysine dihydrate salt at 50 or 100 mg (calculated as the free acid) capsules alone or combined with atorvastatin 20 mg, for 8 weeks) has been reported by Bays et al., “MBX-8025, A Novel Peroxisome Proliferator Receptor-6 Agonist: Lipid and Other Metabolic Effects in Dyslipidemic Overweight Patients Treated with and without Atorvastatin”, J. Clin. Endocrin. Metab., 96(9), 2889-2897 (2011) and Choi et al., “Effects of the PPAR-6 agonist MBX-8025 on atherogenic dyslipidemia”, Atherosclerosis, 220, 470-476 (2012). Compared to placebo, seladelpar alone and in combination with atorvastatin significantly (P<0.05) reduced apoB100 by 20-38%, LDL by 18-43%, triglycerides by 26-30%, non-HDL-C by 18-41%, free fatty acids by 16-28%, and high-sensitivity C-reactive protein by 43-72%; it raised HDL-C by 1-12% and also reduced the number of patients with the metabolic syndrome and a preponderance of small LDL particles. Seladelpar significantly reduced alkaline phosphatase by 32-43%, compared to reductions of only 4% in the control group and 6% in the ATV group; and significantly reduced γ-glutamyl transpeptidase by 24-28%, compared to a reduction of only 3% in the control group and an increase of 2% in the ATV group. Thus seladelpar corrects all three lipid abnormalities in mixed dyslipidemia—lowers TGs and LDL and raises HDL, selectively depletes small dense LDL particles (92%), reduces cardiovascular inflammation, and improves other metabolic parameters including reducing serum aminotransferases (alanine aminotransferase (ALT) and aspartate aminotransferase (AST)), increases insulin sensitivity (lowers HOMA-IR, fasting plasma glucose, and insulin), lowers γ-glutamyl transpeptidase and alkaline phosphatase, significantly (>2-fold) reduces the percentage of subjects meeting the criteria for metabolic syndrome, and trends towards a decrease in waist circumference and increase in lean body mass. Seladelpar was safe and generally well-tolerated, and also reduced liver enzyme levels.

Seladelpar, also as the L-lysine dihydrate salt, has also been studied in primary biliary cholangitis (PBC), with results reported in Jones et al., “Seladelpar (MBX-8025), a selective PPAR-δ agonist, in patients with primary biliary cholangitis with an inadequate response to ursodeoxycholic acid: a double-blind, randomised, placebo-controlled, phase 2, proof-of-concept study”, Lancet Gastroenterol. Hepatol., 2(10), 716-726 (2017), and recently at The International Liver Congress™ hosted by the European Association for the Study of Liver Diseases (EASL) in Paris, France (Apr. 11-15, 2018): posters LBP-2 (Hirschfield et al., “Treatment Efficacy and Safety of Seladelpar, a Selective Peroxisome Proliferator-Activated Receptor Delta agonist, in Primary Biliary Cholangitis Patients: 12- and 26-Week Analyses of an Ongoing, International, Randomized, Dose Ranging Phase 2 Study”) and THU-239 (Boudes et al., “Seladelpar's Mechanism of Action as a Potential Treatment for Primary Biliary Cholangitis and Non-Alcoholic Steatohepatitis”), both available at https://ir.cymabay.com/presentations.

The use of seladelpar and its salts for the treatment of NAFLD and NASH is disclosed in U.S. Pat. Nos. 9,381,181, 9,616,039, and 9,962,346, and Application Publication No. 2018/0228752. Haczeyni et al., “The Selective Peroxisome Proliferator-Activated Receptor-Delta Agonist Seladelpar Reverses Nonalcoholic Steatohepatitis Pathology by Abrogating Lipotoxicity in Diabetic Obese Mice”, Hepatol. Comm., 1(7), 663-674 (2017), have reported that seladelpar improves NASH pathology (reducing hepatic steatosis and inflammation, and improving fibrosis) in atherogenic diet-fed obese diabetic (Alms1 mutant (fozlfoz)) mice, a well-known animal model for human NAFLD/NASH. Choi et al., “Seladelpar Improves Hepatic Steatohepatitis and Fibrosis in a Diet-Induced and Biopsy-Confirmed Mouse Model of NASH”, Abstract 1311 for the Liver Meeting® 2018 of the American Association for the Study of Liver Diseases (AASLD), have reported similar results in atherogenic diet-fed normal (DIO-NASH) mice. CymaBay Therapeutics has initiated a Phase 2b study of seladelpar in patients with NASH using doses of 10, 20, and 50 mg/day, NCT03551522: see CymaBay press release “CymaBay Therapeutics Announces the Initiation of a Phase 2b Study of Seladelpar in Patients with Non-Alcoholic Steatohepatitis”, https://ir.cymabay.com/pres s-releases/detail/431/cymabay-therapeutics-announces-the-initiation-of-a-phase-2b-study-of-seladelpar-in-patients-with-non-alcoholic-steatohepatitis.

The disclosures of the documents referred to in this application are incorporated into this application by reference.

### SUMMARY OF THE INVENTION

This invention is a method of treating NAFLD, including NASH, by concomitant administration of seladelpar or a salt thereof, and a glucagon-like peptide-1 (GLP-1) receptor agonist.

In other aspects, this invention includes:

pharmaceutical compositions for treating NAFLD, including NASH, comprising: seladelpar or a salt thereof, and a GLP-1 receptor agonist; and
kits for treating NAFLD, including NASH, comprising: (a) compositions comprising seladelpar or a salt thereof, and (b) compositions comprising a GLP-1 receptor agonist.

Because concomitant administration of seladelpar (as the L-lysine dihydrate salt) and liraglutide has shown anti-NAFLD/NASH activity in the DIO-NASH mouse model, and because the activity also includes a synergistic effect on obesity in this model, the concomitant administration of seladelpar or a salt thereof and a GLP-1 receptor agonist is expected to show efficacy in the treatment of NAFLD, including NASH.

Preferred embodiments of this invention are characterized by the specification and by the features of claims 1 to 15 of this application as filed.

### DETAILED DESCRIPTION OF THE INVENTION

### Definitions

“NAFLD” and “NASH” and their treatment are described in the sections entitled “NAFLD and NASH” and “Treatments for NAFLD and NASH” in the DESCRIPTION OF THE RELATED ART. Unless the context requires otherwise, reference to NAFLD is a reference both to NAFLD and NASH.

“Seladelpar” is described in the section entitled “Seladelpar” in the DESCRIPTION OF THE RELATED ART

Salts (for example, pharmaceutically acceptable salts) of seladelpar are included in this invention and are useful in the compositions, methods, and uses described in this application. These salts are preferably formed with pharmaceutically acceptable acids. See, for example, “Handbook of Pharmaceutically Acceptable Salts”, Stahl and Wermuth, eds., Verlag Helvetica Chimica Acta, Zurich, Switzerland, for an extensive discussion of pharmaceutical salts, their selection, preparation, and use. Unless the context requires otherwise, reference to seladelpar is a reference both to seladelpar and to its salts.

Because seladelpar contains a carboxyl group, it may form salts when the acidic proton present reacts with inorganic or organic bases. Typically, seladelpar is treated with an excess of an alkaline reagent, such as hydroxide, carbonate or alkoxide, containing an appropriate cation. Cations such as Na⁺, K⁺, Ca²⁺, Mg²⁺, and NH₄⁺ are examples of cations present in pharmaceutically acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. As noted in the DESCRIPTION OF THE RELATED ART, seladelpar is currently formulated as its L-lysine dihydrate salt.

“Glucagon-like peptide-1 (GLP-1) receptor agonists”, are described in the section entitled “GLP-1 receptor agonists” in the DESCRIPTION OF THE RELATED ART. Unless the context requires otherwise, reference to GLP-1 receptor agonists or to each of the GLP-1 receptor agonists, such as liraglutide, is a reference both to the GLP-1 receptor agonist(s) and to its/their salts, if any.

“Concomitant administration” of seladelpar, and a GLP-1 receptor agonist means administration of the seladelpar and the GLP-1 receptor agonist during the course of treatment of NAFLD, including NASH. Such concomitant administration may involve administration of the GLP-1 receptor agonist before, during, and/or after administration of the seladelpar, such that therapeutically effective levels of each of the compounds are maintained during the treatment. Because most of the GLP-1 receptor agonists are administered by injection at different frequencies, concomitant administration will be accomplished by administration of the seladelpar daily and the GLP-1 receptor agonist at its usual dosing; but concomitant administration of an orally administrable GLP-1 receptor agonist such as semaglutide may include the administration of the seladelpar and the GLP-1 receptor antagonist daily and may also include administration of a combination oral dosage form containing both the seladelpar and the GLP-1 receptor agonist. “Combination therapy” with seladelpar and a GLP-1 receptor agonist has the same meaning as “concomitant administration”.

A “therapeutically effective amount” of seladelpar, or of a GLP-1 receptor agonist administered concomitantly with the seladelpar, means that amount which, when the seladelpar and the GLP-1 receptor agonist are concomitantly administered to a human for treating NAFLD, including NASH, is sufficient to effect treatment for the NAFLD or NASH. “Treating” or “treatment” of NAFLD, including NASH, in a human includes one or more of: (1) preventing or reducing the risk of developing NAFLD or NASH, i.e., causing the clinical symptoms of NAFLD or NASH not to develop in a subject who may be predisposed to NAFLD or NASH but who does not yet experience or display symptoms of the NAFLD or NASH (i.e. prophylaxis);

(2) inhibiting NAFLD or NASH, i.e., arresting or reducing the development of NAFLD or NASH or its clinical symptoms; and
(3) relieving NAFLD or NASH, i.e., causing regression, reversal, or amelioration of the NAFLD or NASH or reducing the number, frequency, duration or severity of its clinical symptoms. The therapeutically effective amount for a particular subject varies depending upon the health and physical condition of the subject to be treated, the extent of the NAFLD or NASH, the assessment of the medical situation, and other relevant factors. It is expected that the therapeutically effective amount will fall in a relatively broad range that can be determined through routine trial.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

Formulation and Administration

The seladelpar and the GLP-1 receptor agonist may be concomitantly administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because seladelpar is orally available, typical formulations will be oral, and typical dosage forms of the seladelpar component of the combination therapy, or of the two components separately or together if the GLP-1 receptor agonist is orally administrable, will be tablets or capsules for oral administration. Most of the GLP-1 receptor agonists are, at the moment, formulated as solutions for subcutaneous injection, dispensed in prefilled multi-dose syringe “pen”-type injectors; but an oral formulation of semaglutide has been approved in the United States and oral formulations of other GLP-1 receptor agonists are therefore expectable and may be used in the practice of this invention.

Depending on the intended mode of administration, the pharmaceutical compositions may be in the form of solid, semi-solid or liquid dosage forms, preferably in unit dosage form suitable for single administration of a precise dosage. In addition to an effective amount of the seladelpar and/or the GLP-1 receptor agonist, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule, or it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of seladelpar is packaged in a container with a label, or instructions, or both, indicating use of the pharmaceutical composition in the treatment of NAFLD and/or NASH. Typically, a pharmaceutical composition of the combination of seladelpar and an orally-administrable GLP-1 receptor agonist, or a kit comprising separate compositions of seladelpar and of a GLP-1 receptor agonist, is packaged in a container with a label, or instructions, or both, indicating use of the pharmaceutical composition or kit in the treatment of NAFLD and/or NASH.

A person of ordinary skill in the art of pharmaceutical formulation will be able to prepare suitable pharmaceutical compositions of the seladelpar and the GLP-1 receptor agonist, and of oral combinations of seladelpar and an orally-administrable GLP-1 receptor agonist, by choosing suitable dosage forms, excipients, packaging, and the like, to achieve therapeutically effective formulations without undue experimentation and in reliance upon personal knowledge and the disclosure of this application.

A suitable amount of seladelpar or a salt thereof (calculated as seladelpar) for oral dosing when administered alone (i.e. not administered in combination with a GLP-1 receptor agonist: NAFLD/NASH patients may well be taking other therapies in addition to the seladelpar and GLP-1 receptor agonists discussed in this application) is expected to be 5-200 mg/day, preferably 10-100 mg/day, such as 10, 20, 50, or 100 mg/day. That is, a suitable amount of seladelpar for oral dosing is expected to be similar to the amounts employed in clinical trials for NASH and other conditions. Suitable reductions in dose toward the lower end of the outer range above will be made for subjects who are children, depending on such additional factors as age and body mass.

When seladelpar and a GLP-1 receptor agonist are concomitantly administered, a suitable amount of seladelpar is expected to be the same as when seladelpar is administered alone; and a suitable amount of the GLP-1 receptor agonist is expected to be similar to the amount approved or used in clinical trials, as described in the section entitled “GLP-1 receptor agonists” in the DESCRIPTION OF THE RELATED ART. Thus, for example, a suitable amount of liraglutide for subcutaneous dosing is expected to be between 1 and 2 mg/day, such as 1.2 and 1.8 mg/day, while a suitable amount of semaglutide for oral dosing is expected to be between 5 and 40 mg/day, such as 7 or 14 mg/day. However, it is possible that the therapeutically effective amounts of either may be less in combination therapy than when used as monotherapy because each of them is expected to possess some efficacy in treating NAFLD/NASH.

A person of ordinary skill in the art of the treatment of NAFLD/NASH will be able to ascertain a therapeutically effective amount of seladelpar and GLP-1 receptor agonist, when used by concomitant administration, for a particular patient and stage of NAFLD, including NASH, to achieve a therapeutically effective amount without undue experimentation and in reliance upon personal knowledge and the disclosure of this application.

### EXAMPLES

### Example 1 (Pre-Clinical, Concomitant Administration with Single Agent Seladelpar Comparison)

The diet-induced obese mouse model of NASH (DIO-NASH) uses the C57BL/6J mouse fed a high fat diet that results in NAFLD/NASH. A protocol is described in Kristiansen et al., “Obese diet-induced mouse models of nonalcoholic steatohepatitis—tracking disease by liver biopsy”, World J. Hepatol., 8(16), 673-684 (2016). Male C57BL/6J mice were fed an atherogenic 40% high fat diet (AMLN diet, D09100301, Research Diet, US—40 kcal % fat (18% trans fat), 40 kcal % carbohydrate (20% fructose), 2% cholesterol) for 43 weeks before the start of the trial, to induce NAFLD/NASH. At week-3, the mice underwent a liver biopsy, which was scored for steatosis and fibrosis; mice with fibrosis stage <1 and steatosis score <2 were deselected prior to randomization. A stratified randomization into treatment groups was performed according to liver Collα1 quantification. The mice were then continued on the same diet and dosed with vehicle (1% methylcellulose, once/day), seladelpar (10 mg/Kg in vehicle once/day), liraglutide (0.2 mg/Kg, subcutaneously twice/day), or seladelpar and liraglutide, with obeticholic acid (30 mg/Kg in vehicle once/day) as positive control, for 12 weeks. At 12 weeks, analyses included plasma ALT, AST, triglycerides, and total cholesterol; liver triglycerides and total cholesterol; and NAS, fibrosis, Collα1, galectin-3, and steatosis and fibrosis scores from a liver biopsy. Results are given in the table below: standard deviations are in parentheses:

| | | Seladelpar | Liraglutide | |
|---|---|---|---|---|
| Treatment | Vehicle | (S) | (L) | S + L |
| Number of animals | 12 | 11 | 11 | 12 |
| Body weight | 100 (3) | 91 (6) | 89 (6) | 82 (6) |
| (% relative to | | | | |
| vehicle) | | | | |
| ALT (U/L) | 270 (94) | 107 (65) | 67 (32) | 39 (15) |
| AST (U/L) | 279 (98) | 162 (59) | 122 (34) | 75 (13) |
| Plasma TG (mg/dL) | 62 (14) | 38 (20) | 42 (17) | 24 (11) |
| Plasma TC (mg/dL) | 317 (46) | 257 (40) | 200 (40) | 191 (46) |
| Liver TG (mg/g liver) | 98 (21) | 75 (20) | 77 (26) | 54 (17) |
| Liver TC (mg/g liver) | 12 (3) | 10 (3) | 10 (2) | 8 (2) |
| Sirius red stain (%) | 3.7 (2.1) | 2.4 (1.8) | 3.4 (2.1) | 2.0 (1.3) |
| Steatosis (% relative | 0.1 (15) | −56 (23) | −43 (25) | −76 (8) |
| to baseline) | | | | |
| NAS pre-treatment | 5.5 (0.7) | 6.0 (0.8) | 6.1 (0.8) | 5.7 (0.5) |
| NAS post-treatment | 5.8 (0.7) | 3.9 (0.5) | 4.6 (0.8) | 3.1 (0.7) |

### Example 2A (Pre-Clinical, Single Agent Seladelpar—Haczeyni et al.)

From weaning, female Alms1 mutant (fozlfoz) mice and wild-type littermates were fed an atherogenic diet for 16 weeks; groups (n=8-12) were then randomized to receive seladelpar (10 mg/Kg in vehicle) or vehicle (1% methylcellulose) by gavage for 8 weeks. Despite minimally altering body weight, seladelpar normalized hyperglycemia, hyperinsulinemia, and glucose disposal in fozlfoz mice. Serum alanine aminotransferase ranged from 300-600 U/L in vehicle-treated fozlfoz mice; seladelpar reduced alanine aminotransferase by 50%. In addition, seladelpar normalized serum lipids and hepatic levels of free cholesterol and other lipotoxic lipids that were increased in vehicle-treated fozlfoz versus wild-type mice. This abolished hepatocyte ballooning and apoptosis, substantially reduced steatosis and liver inflammation, and improved liver fibrosis. In vehicle-treated fozlfoz mice, the mean nonalcoholic fatty liver disease activity score (NAS) was 6.9, indicating NASH; seladelpar reversed NASH in all fozlfoz mice (NAS 3.13).

### Example 2B (Pre-Clinical, Concomitant Administration)

The methods of Example 2A are followed, except that instead of dosing only with seladelpar or vehicle, further groups of fozlfoz mice are dosed with chosen GLP-1 receptor agonists individually, such as with liraglutide, and with combinations of seladelpar and a GLP-1 receptor agonist, such as seladelpar and liraglutide. The mice show dose-related and combination-related improvement in their disease.

### Example 3 (Clinical, Single Agent Seladelpar)

One hundred seventy-five subjects with liver biopsy-proven NASH are treated with doses of 10, 20, and 50 mg/day, or placebo (2:2:2:1 randomization) for 52 weeks. Subjects are permitted their usual other medications (e.g. antidiabetic medications such as metformin or sulfonamides) but not glitazones, PPAR agonists, OCA, or similar medications. The subjects are assessed before the study, and at intervals during the study, such as every 4 weeks during the study and 4 weeks after the last dose of the seladelpar therapy, for safety and pharmacodynamic evaluations.

The primary efficacy outcome is the change in baseline in liver fat content at 12 weeks, as measured by magnetic resonance imaging-derived proton density fat fraction (MRI-PDFF). Other outcome measures include histological improvement in NASH and fibrosis, assessed by comparing liver biopsy samples at baseline and at 52 weeks after the start of dosing; MRI-PDFF at 26 and 52 weeks; and measurements of total cholesterol, HDL-C, LDL-C, VLDL-C, TGs, apoB, and liver transaminases. The subjects also maintain health diaries, which are reviewed at each visit. The subjects show a dose-related improvement in their disease, as manifested by, for example, MRI-PDFF and liver biopsy, and improvement in components of, and total, NAS score.

### Example 4 (Clinical, Concomitant Administration)

The methods of Example 3 are followed, except that instead of dosing only with seladelpar or placebo, further groups of subjects are dosed concomitantly with seladelpar and a GLP-1 receptor agonist, such as seladelpar and liraglutide, seladelpar and semaglutide, seladelpar and tirzepatide, etc., using daily dosing of seladelpar and dosing of the GLP-1 receptor agonist according to its usual dose and dose frequency tested for NASH or tested or approved for T2DM. The subjects show dose-related and combination-related improvement in their disease.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating non-alcoholic fatty liver disease, comprising concomitant administration of a therapeutically effective amount of:
(a) seladelpar or a salt thereof; and
(b) a glucagon-like peptide-1 (GLP-1) receptor agonist.

2. The method of claim 1 where the seladelpar or a salt thereof is a seladelpar L-lysine salt.

3. The method of claim 2 where the seladelpar or a salt thereof is seladelpar L-lysine dihydrate salt.

4. The method of claim 1 where the seladelpar or a salt thereof is administered orally.

5. The method of claim 1 where the daily dose of the seladelpar or a salt thereof is 5-200 mg, when the dose is calculated as seladelpar.

6. The method of claim 5 where the daily dose of the seladelpar or a salt thereof is 10-100 mg.

7. The method of claim 6 where the daily dose of the seladelpar or a salt thereof is 10-50 mg.

8. The method of claim 7 where the daily dose of the seladelpar or a salt thereof is 10, 20, or 50 mg/day.

9. The method of claim 1 where the seladelpar or a salt thereof is administered once/day.

10. The method of claim 1 where the non-alcoholic fatty liver disease is non-alcoholic steatohepatitis.

11. The method of claim 1 where the GLP-1 receptor agonist is liraglutide, semaglutide, exenatide, lixisenatide, dulaglutide, or tirzepatide.

12. The method of claim 11 where the GLP-1 receptor agonist is liraglutide or semaglutide.

13. The method of claim 11 where the GLP-1 receptor agonist is tirzepatide.

14. An oral pharmaceutical composition containing:
seladelpar or a salt thereof, and
a GLP-1 receptor agonist.

15. The oral pharmaceutical composition of claim 14 where the GLP-1 receptor agonist is semaglutide.
